SEQUENCE LISTING (10 sequences)

SEQ ID NO: 1 - DNA, 20 bp, Artificial sequence
  Synthetic oligonucleotide primer to the L1 transposable element.
aaagaccccaacgagaagcg

SEQ ID NO: 2 - DNA, 23 bp, Artificial sequence
  Synthetic oligonucleotide primer to the L1 transposable element.
cctattggcgttactatgggaac

SEQ ID NO: 3 - DNA, 23 bp, Artificial sequence
  Synthetic oligonucleotide primer to EGFP.
gcaccatcttcttcaaggacgac

SEQ ID NO: 4 - DNA, 20 bp, Artificial sequence
  Synthetic oligonucleotide primer to EGFP.
tctttgctcagggcggactg

SEQ ID NO: 5 - DNA, 20 bp, Artificial sequence
  Synthetic oligonucleotide primer to the mouse BRCA1 gene.
aagccaaaacacatagaccc

SEQ ID NO: 6 - DNA, 20 bp, Artificial sequence
  Synthetic oligonucleotide primer to the mouse BRCA1 gene.
ctgcctgtcgttacatgttc

SEQ ID NO: 7 - DNA, 20 bp, Artificial sequence
  Synthetic oligonucleotide primer to the mouse HBA1 gene.
tgagccaggaagtgtgtgtg

SEQ ID NO: 8 - DNA, 20 bp, Artificial sequence
  Synthetic oligonucleotide primer to the mouse HBA1 gene.
aggatggaagcagtctccag

SEQ ID NO: 9 - DNA, 21 bp, Artificial sequence
  Synthetic oligonucleotide primer to the mouse MSH2 gene.
tccctcactttgatgtaagcc

SEQ ID NO: 10 - DNA, 20 bp, Artificial sequence
  Synthetic oligonucleotide primer to the mouse MSH2 gene.
ccaagaggtacaggtgcaag

(19) United States
(12) Patent Application Publication
(10) Pub. No.: US 2010/0184152 A1
(43) Pub. Date: Jul. 22, 2010

(54) TARGET-ORIENTED WHOLE GENOME AMPLIFICATION OF NUCLEIC ACIDS

(76) Inventors: Vladislav Sandler, Melrose, MA (US); Fred H. Gage, La Jolla, CA (US)

Correspondence Address: KLARQUIST SPARKMAN, LLP, 121 S.W. SALMON STREET, SUITE 1600, PORTLAND, OR, 97204, US

(21) Appl. No.: 12/445,926

(22) PCT Filed: Oct. 22, 2007
(86) PCT No.: PCT/US07/82135

§ 371 Date: Apr. 16, 2009

Related U.S. Application Data
(60) Provisional application No. 60/862,479, filed on Oct. 23, 2006.

(51) Int. Cl. C12P 19/34; C12N 9/10

(52) U.S. Cl. 435/91.2; 435/193

(57) ABSTRACT

Disclosed herein are methods of amplifying target nucleic acid sequences (e.g., DNA or RNA), particularly from a very small amount of starting material, such as a single cell. These methods involve targeting the amplification of specific sequence(s) by use of sequence-specific primers and random primers for whole genome amplification using multiple displacement amplification. Generally, the provided methods are referred to herein as “target-oriented” whole genome amplification. Starting material for target-oriented whole genome amplification can be any sample containing DNA or RNA, however, the technique is particularly suitable for very small amounts of starting material, such as a few cells, a single cell, or a single nucleus. The methods provide amplified nucleic acid (including the target sequence of interest) that can subsequently be analyzed.

### CROSS-REFERENCE TO RELATED APPLICATIONS

This application claims the benefit of U.S. Provisional Application No. 60/862,479 filed Oct. 23, 2006, herein incorporated by reference.

### FIELD

This disclosure relates to methods for amplifying target nucleic acid sequences (e.g., DNA or RNA), particularly from a very small amount of starting material, such as a single cell.

### BACKGROUND

A number of methods have been described for whole genome amplification (WGA) from samples where only a small amount of starting material is available. These methods include polymerase chain reaction (PCR)-based methods such as degenerate oligonucleotide primed PCR (DOP-PCR), primer extension preamplification PCR (PEP-PCR), and ligation-mediated PCR. One drawback of PCR-based WGA methods is that they generate low molecular weight (“short”) DNA that may not be suitable for downstream analysis. An isothermal method of WGA, multiple displacement amplification (MDA), has also been described (Dean et al. Proc. Natl. Acad. Sci. 99:5261-5266, 2002; U.S. Pat. No. 6,124,120; U.S. Pat. No. 6,977,148). MDA uses random primers and a DNA polymerase with strand displacement activity. This leads to multiple priming events, as newly synthesized DNA is displaced from the template, forming a network of branched structures. An advantage of MDA is synthesis of high molecular weight (“long”) DNA products.

All of the currently available WGA techniques have the limitation of generating DNA with incomplete coverage of loci throughout the genome (sometimes referred to as allelic dropout (ADO)), particularly when little starting material, for example, a single cell, is used (Spits et al. Hum. Mut. 27:496-503, 2006). ADO rates from single-cell WGA, whether by MDA or PCR-based methods, range from 25 to 33% (Spits et al.). As a result, following conventional WGA, by MDA or other techniques, subsequent analysis of genes or markers of interest is not possible for up to one-third of the genome. Another disadvantage of MDA is template-independent background DNA synthesis, which may be the result of the random hexamer primers in the reaction becoming the template for Phi29 polymerase (Holbrook et al. J. Biomol. Tech. 16:125-133, 2005).

### SUMMARY

Methods of nucleic acid (e.g., DNA or RNA) amplification are provided. In particular examples, the method permits whole genome amplification (WGA) and provides reliable representation of sequences of interest following multiple displacement amplification (referred to as “target-oriented WGA,” or TOWGA). The disclosed methods can be used to ensure that specific sequences of interest (that is, targets) are present for further analysis following amplification of a sample.

In certain embodiments, TOWGA involves adding one primer designed to be complementary to one specific sequence of interest to the set of random primers used for MDA. As a result, the sequence of interest is reliably amplified in the sample, which also contains sequences amplified from the random primers in the primer set.

In some examples, the provided method permits targeting of multiple sequences d in the context of one TOWGA amplification reaction. Thus, in certain examples, more than one sequence-specific primer is included in the primer set, each of which is designed to be complementary to a target to be maintained in the amplification (for instance, for downstream analysis). By way of example, the multiple sequence-specific primers may be complementary to multiple genes or other sequences of interest, multiple regions of a single gene or of several genes or sequences, or a single region of a nucleic acid molecule.

TOWGA is suitable for the reliable amplification of target sequences even from a very small amount of starting material. For example, samples containing only a few cells, a single cell, or even the nucleus from a single cell, can be used as starting material for TOWGA.

In certain embodiments, the target-oriented amplification method is repeated using amplified DNA from a first round of TOWGA. This enables generation of greater amounts of amplified DNA, while maintaining in the amplified sample the target(s) for which specific primers have been included in the primer set. In other embodiments, a replicated DNA (or amplified DNA) sample generated using TOWGA is subjected to conventional nucleic acid amplification.

DNA amplified using TOWGA can be used for downstream analysis, for instance by any conventional molecular biology techniques, such as PCR and DNA sequencing.

In one specific embodiment, there is provided a method for amplifying a specific sequence of interest from a sample, which method involves incubating the sample (which contains nucleic acid to serve as template, e.g., DNA or RNA) and a set of primers including random primers and a sequence-specific primer, with a DNA or RNA polymerase under conditions that promote strand displacement replication. Optionally, nucleic acid amplicons obtained from the target-oriented amplification reaction may be used as starting material for a second TOWGA reaction in order to generate additional nucleic acid molecules for downstream analysis.

In another specific embodiment, there is provided a method for amplifying multiple specific sequences of interest from a sample (which contains template nucleic acid e.g., DNA or RN), which method involves incubating the sample with a set of primers including random primers, multiple sequence-specific primers (each of which is designed to be complementary to a sequence of interest), and a DNA or RNA polymerase under conditions that promote strand displacement replication.

The foregoing and other features will become more apparent from the following detailed description, which proceeds with reference to the accompanying figures.

### BRIEF DESCRIPTION OF THE DRAWINGS

FIG. 1 is a digital image of a gel showing PCR products obtained following TOWGA using single mouse nuclei as starting material. Each lane shows products from PCR using GFP primers following TOWGA on a single nucleus. The 1243 by product indicates the presence of the L1 element transgene. The 343 by product indicates the integration of the transgene and the expression of the GFP gene.

FIG. 2 is a digital image of a gel showing PCR products obtained following TOWGA or WGA using a single mouse neuron nucleus as starting material. Lanes 1 and 2 show PCR using GFP primers from two different nuclei from mouse brain that expressed GFP, as detected by fluorescence. The nuclei were subjected to a round of WGA, followed by a round of TOWGA which included GFP-specific primers (2° TOWGA) prior to PCR. Lanes 3 and 4 show GFP PCR products obtained from the same mouse neuron nuclei as lanes 1 and 2. Following the round of WGA (as in lanes 1 and 2), these samples were subjected to a second round of WGA (2° WGA) prior to PCR with GFP-specific primers. The 1243 by product indicates the presence of the L1 element transgene. The 243 by product indicates the integration of the transgene and the expression of the GFP gene.

FIG. 3 is a digital image of a gel showing PCR products obtained from IPCR following the 2° TOWGA shown in Lanes 1 and 2 of FIG. 1, respectively. Products shown by wows were cut from the gel, purified and sequenced to identify the location of the retrotransposed L1 element in a given cell.

FIG. 4 shows results of TOWGA with simultaneous inclusion of multiple sequence-specific primers. FIG. 4A is a digital image of a gel showing PCR products obtained following TOWGA using a single mouse neuron nucleus as starting material. A round of 1° WGA was followed by TOWGA using multiple sequence-specific primers. TOWGA amplified DNA was then used for PCR reactions containing sets of gene-specific primers. Lane 1: BRCA1; Lane 2: HBA1; Lane 3: MSH2; Lane 4: molecular weight marker. FIG. 4B shows representative sequence obtained from the PCR product obtained using HBA1 primers shown in FIG. 4A, confirming the identity of the PCR product.

### BRIEF DESCRIPTION OF THE SEQUENCE LISTING

The nucleic and amino acid sequences listed in the accompanying sequence listing are shown using standard letter abbreviations for nucleotide bases, and three-letter code for amino acids, as defined in 37 C.F.R. §1.822. Only one strand of each nucleic acid sequence is shown, but the complementary strand is understood as included by any reference to the displayed strand. In the accompanying sequence listing:

SEQ ID NOs: 1 and 2 are oligonucleotide primers complementary to the L1 transposable element.

SEQ ID NOs: 3 and 4 are oligonucleotide primers complementary to EGFP.

SEQ ID NOs: 5 and 6 are oligonucleotide primers complementary to the mouse BRCA1 gene.

SEQ ID NOs: 7 and 8 are oligonucleotide primers complementary to the mouse HBA1 gene.

SEQ ID NOs: 9 and 10 are oligonucleotide primers complementary to the mouse MSH2 gene.

### DETAILED DESCRIPTION

### I. Abbreviation

GFP green fluorescent protein

IPCR inverse PCR

IVF: in vitro fertilization

PCR: polymerase chain reaction

PGD: pre-implantation genetic diagnosis

MDA: multiple displacement amplification

TOWGA: target-oriented whole genome amplification

WGA: whole genome amplification

### II. Terms

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “including a nucleic acid” encompasses single or plural nucleic acids, and is considered equivalent to the phrase “including at least one nucleic acid.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. For example, the phrase “mutations or polymorphisms” or “one or more mutations or polymorphisms” means a mutation, a polymorphism, or combinations thereof, wherein “a” can refer to more than one.

Although methods and materials similar or equivalent to those described herein can be used in the practice or testing of the present disclosure, suitable methods and materials are described below. The materials, methods, and examples are illustrative only and not intended to be limiting.

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

Amplification: An increase in the amount of (number of copies of) nucleic acid sequence, wherein the increased sequence is the same as or complementary to the nucleic acid template. An example of amplification is the polymerase chain reaction (PCR), in which a sample containing nucleic acid template is contacted with a pair of oligonucleotide primers (one upstream to the target sequence, the other downstream and on the opposing strand), under conditions that allow for the hybridization (annealing) of the primers to nucleic acid template in the sample. The primers are extended under suitable conditions (though nucleic acid polymerization). If additional copies of the nucleic acid are desired, the first copy is dissociated from the template, and additional copies of the primers (usually contained in the same reaction mixture) are annealed to the template, extended, and dissociated repeatedly to amplify the desired number of copies of the nucleic acid.

The products of amplification may be characterized by myriad techniques, including for instance electrophoresis, restriction endonuclease cleavage patterns, hybridization, nucleic acid sequencing, and other techniques known in the art.

Other examples of amplification techniques include reverse-transcription PCR(RT-PCR); strand displacement amplification (see U.S. Pat. No. 5,744,311); transcription-free isothermal amplification (see U.S. Pat. No. 6,033,881); repair chain reaction amplification (see WO 90/01069); ligase chain reaction amplification (see EP-A-320 308); gap filling ligase chain reaction amplification (see U.S. Pat. No. 5,427,930); coupled ligase detection and PCR (see U.S. Pat. No. 6,027,889); and NASBAT™ RNA transcription-free amplification (see U.S. Pat. No. 6,025,134).

Further examples of amplification techniques include methods of whole genome amplification, such as degenerate oligonucleotide primed PCR (DOP-PCR), primer extension preamplification PCR (PEP-PCR), ligation-mediated PCR, and multiple displacement amplification (MDA).

Complementary: One nucleic acid molecule is complementary with another nucleic acid molecule if the two molecules share a sufficient number of complementary nucleotides to form a stable duplex or triplex when the strands bind (hybridize) to each other, for example by forming Watson-Crick, Hoogsteen or reverse Hoogsteen base pairs. Stable binding occurs when a nucleic acid molecule (e.g., a sequence-specific primer) remains detectably bound to a target nucleic acid sequence (e.g., a genomic target nucleic acid sequence) under the required conditions.

Complementarity is the degree to which bases in one nucleic acid molecule (e.g., a sequence-specific primer) base pair with the bases in a second nucleic acid molecule (e.g., a genomic target nucleic acid sequence). Complementarity is conveniently described by percentage, that is, the proportion of nucleotides that form base pairs between two molecules or within a specific region or domain of two molecules. For example, if 10 nucleotides of a 15 by sequence-specific primer form base pairs with a target nucleic acid molecule, that sequence-specific primer is said to have 66.67% complementarity to the target nucleic acid molecule.

In the present disclosure, “sufficient complementarity” means that a sufficient number of base pairs exist between one nucleic acid molecule or region thereof (such as a sequence-specific primer) and a target nucleic acid sequence (e.g., genomic target nucleic acid sequence) to achieve detectable binding. A thorough treatment of the qualitative and quantitative considerations involved in establishing binding conditions is provided by Beltz et al. Methods Enzymol. 100:266-285, 1983, and by Sambrook et al. (ed.), Molecular Cloning: A Laboratory Manual, 2nd ed., vol. 1-3, Cold Spring Harbor Laboratory Press, Cold Spring Harbor, N.Y., 1989.

Embryo: An organism in the early stages of growth and differentiation that is characterized by cleavage, the laying down of fundamental tissues, and the formation of primitive organs and organ systems. In humans, the term embryo is used to describe developmental stages from the time of implantation to about the end of the eighth week after conception. A fertilized egg that has begun cell division, but has not yet implanted, is a pre-embryo. The pre-embryonic stage is generally considered to end at about day 14 of development in humans.

Fetus/Fetal: An unborn or unhatched organism in later stages of growth and differentiation, having the basic structural resemblance to the adult animal, especially after the appearance of the first bone cells. In humans, it is the period from after about eight weeks of development until birth.

Forensic sample: A sample that may be used for the application of science or technology in the investigation and establishment of facts or evidence, for instance for use in a court of law. A forensic sample is often a sample taken from a non-biological source that is used to extract biological material that may be used for the isolation and analysis of DNA or RNA. One example of a forensic sample is a piece of carpet that contains drops of blood. The blood may be extracted from the carpet, such as by collection with a swab, and DNA or RNA can subsequently be isolated using standard techniques. Examples of biological materials that may be used for forensic testing include, but are not limited to, blood, saliva, semen, urine or feces, hair, skin, bone, and other body tissues.

Genome: The total genetic constituents of an organism. In the case of eukaryotic organisms, the genome is contained in a haploid set of chromosomes of a cell. In the case of prokaryotic organisms, the genome is contained in a single chromosome, and in some cases one or more extra-chromosomal genetic elements, such as episomes (e.g., plasmids). A viral genome can take the form of one or more single or double stranded DNA or RNA molecules depending on the particular virus.

In vitro fertilization (IVF): Fertilization of an egg in vitro, for example in a laboratory dish or test tube. IVF is usually accomplished by the mixture, usually in a laboratory dish, of sperm with eggs that have been surgically removed from a donor female.

Modified nucleotide (modified nucleoside triphosphate): A modified nucleotide is a nucleotide that has been altered, for example a nucleotide to which a chemical moiety has been added, often one that gives an additional functionality to the modified nucleotide. Generally, the modification comprises a functional group or a leaving group, such as permits coupling of the nucleotide to a detectable molecule, e.g., a fluorophore or hapten. The term also includes nucleotides containing a modified base, a modified sugar moiety, and/or a modified phosphate backbone, for example as described in U.S. Pat. No. 5,866,336.

Examples of modified sugar moieties which may be used at any position on its structure to modify a nucleotide include, but are not limited to: arabinose, 2-fluoroarabinose, xylose, and hexose. A modified component of the phosphate backbone includes, but is not limited to, a phosphorothioate, a phosphorodithioate, a phosphoramidothioate, a phosphoramidate, a phosphordiamidate, a methylphosphonate, an alkyl phosphotriester, or a formacetal or analog thereof.

Multiple displacement amplification (MDA): A method of replication (or amplification) of DNA that utilizes the strand displacement activity of certain DNA polymerases. The method generally involves hybridization of primers, for example random primers, such as random hexamers, to a template nucleic acid sequence, and replication of the sequence. During replication, the elongating strands displace other strands from the template sequence (or from another replicated strand) by strand displacement replication. Strand displacement replication refers to DNA replication (polymerization) where a growing end of a replicated strand encounters and displaces another strand from the template strand or another replicated strand. See U.S. Pat. Nos. 6,124,120 and 6,977,148, for instance.

Phi29 DNA polymerase: A DNA polymerase from the bacteriophage Phi29. See U.S. Pat. No. 5,198,543, for instance. Phi29 DNA polymerase has a 3′-5′ exonuclease (proofreading) activity, but lacks 5′-3′ exonuclease activity. This polymerase is highly processive, and has the ability to generate replicated strands of up to about 70 kb. Further, Phi29 DNA polymerase has the ability to displace the non-template DNA strand of a double-stranded DNA molecule and to continue synthesis along the thereby exposed template strand. This property allows DNA amplification using Phi29 polymerase to be carried out isothermally, that is, without the need for temperature cycling (which is used to dissociate double-stranded DNA in, for example, PCR).

Primer: Primers are relatively short nucleic acid molecules (oligonucleotides), usually DNA oligonucleotides six nucleotides or more in length. Primers can be annealed to a complementary target DNA strand (“priming”) by nucleic acid hybridization to form a hybrid between the primer and the target DNA strand, and then the primer can be extended along the target DNA strand by a nucleic acid polymerase enzyme. A single primer can be used for amplification of a nucleic acid sequence in some methods, e.g. by MDA or TOWGA. Pairs of primers are used for amplification of a nucleic acid sequence in other methods, e.g., PCR.

A random primer is a primer with a random sequence (see, for instance, U.S. Pat. Nos. 5,043,272 and 5,106,727). “Random” sequence in this context means that the positions of alignment and binding (annealing) of the primers to a template nucleic acid molecule are substantially indeterminate with respect to the template under conditions wherein the primers are used to initiate polymerization of a complementary nucleic acid. Methods for estimating the frequency at which an oligonucleotide of a certain sequence will appear in a nucleic acid polymer are described in Volinia et al. (Comp. App. Biosci., 5: 33-40, 1989).

The sequences of random primers may not be random in the absolute mathematic sense. For instance, chemically synthesized random primers will be random to the extent that physical and chemical efficiencies of the synthetic procedure will allow, and based on the method of synthesis. Random primers derived from natural sources (e.g., through digestion of an existing polynucleotide) may be less random, due to favored or disfavored arrangements of bases in the source organism. Oligonucleotides having defined sequences may satisfy the concept of being random if the conditions of their use cause the locations of their apposition to the template to be indeterminate. Also, random primers may be “random” over only a portion of their length, in that one residue within the primer sequence, or a portion of the sequence, can be identified and defined prior to synthesis of the primer.

Random primers may be generated using available oligonucleotide synthesis procedures; randomness of the sequence in some examples is introduced by providing a mixture of nucleic acid residues in the reaction mixture at one or more addition steps (to produce a mixture of oligonucleotides with random sequence). Thus, a random primer can be generated by sequentially incorporating nucleic acid residues from a mixture of 25% of each of dATP, dCTP, dGTP, and dTTP, to form an oligonucleotide. Other ratios of dNTPs can be used (e.g., more or less of any one dNTP, with the other proportions adapted so the whole amount is 100%).

The term “random primer” specifically includes a collection of individual oligonucleotides of different ‘sequences, for instance which can be indicated by the generic formula 5′-XXXXXX-3’, wherein X represents a nucleotide residue (or modified nucleotide residue) that was added to the oligonucleotide from a mixture of a definable percentage of each dNTP. For instance, if the mixture contained 25% each of dATP, dCTP, dGTP, and dTTP, the indicated primer would contain a mixture of oligonucleotides that each have a roughly 25% average chance of having A, C, G, or T at each position. Random primers may contain modified nucleotides, such as nucleotides containing a modified base, a modified sugar moiety, and/or a modified phosphate backbone.

A sequence-specific primer, as used herein, is a primer that is designed to be complementary to a particular sequence of interest (a target sequence), or a sequence adjacent to a sequence of interest. Sequence-specific primers are designed to hybridize to, and prime replication of, a specific sequence that is to be maintained in an amplification reaction, and in many instances the specific sequence is targeted for further analysis. Sequence-specific primers are generally 5 to 60 nucleotides in length, in some instances are 15 to 30 nucleotides in length, or about 20 to 23 nucleotides in length. Sequence-specific primers may contain modified nucleotides, such as nucleotides containing a modified base, a modified sugar moiety, and/or a modified phosphate backbone.

Replication: The process of duplicating or reproducing, such as the replication of a copy of a polynucleotide strand of DNA or RNA. Replication of DNA may be accomplished using any of a number of DNA polymerases (or mixtures thereof), including, but not limited to, DNA polymerase I, T4 or T7 DNA polymerase, Taq DNA polymerase, Phi29 DNA polymerase, Bst DNA polymerase, Vent_{R}® and Deep Vent_{R}® DNA polymerases, 9° N. DNA polymerase, Klenow fragment of DNA polymerase I, PhiPRD1 DNA polymerase, phage M2 DNA polymerase, T4 DNA polymerase, and T5 DNA polymerase. RNA is likewise replicated by an RNA polymerase, including, but not limited to, RNA polymerase II, T7 RNA polymerase, and SP6 RNA polymerase. Other polymerases are available, as will be recognized by one of skill in the art.

Isothermal replication is replication that is not dependent on significant changes in temperature (in contrast to PCR, for example). Thus, it is carried out substantially at about the same single temperature, without thermal cycling.

Sample: A source of one or more nucleic acid molecules (e.g., DNA or RNA), such as material from an animal or plant source. Samples include biological samples such as those derived from a human or other animal source (for example, blood, stool, sera, urine, saliva, tears, tissue biopsy samples, surgical specimens, histology tissue samples, autopsy material, cellular smears, embryonic or fetal cells, amniocentesis or chorionic villus samples, etc.); bacterial or viral or other microbial preparations; cell cultures; forensic samples; agricultural products; waste or drinking water, milk or other processed foodstuff; air; and so forth. Samples suitable for disclosed methods include nucleic acid molecules (e.g., DNA or RNA).

A sample can contain multiple cells, a single cell, no intact cells at all, or can be prepared from cells, such as from a single cell, for instance a nucleus. Samples of limited quantity are contemplated, such as biopsies (such as tumor biopsies), forensic samples, archived DNA or tissue samples, and embryo biopsies and other embryo and pre-embryo samples (such as cells from an in vitro fertilization). Samples containing a small number of cells, or a single cell, can be acquired by any one of a number of methods, such as fine needle aspiration, micro-dissection, biopsy, tissue scrapes, forensic swabs, or laser capture micro-dissection. Samples can also be diluted to a level where they contain as few as 100 cells, ten cells, or even as few as one cell in a sample, and used e.g., for subsequent analysis. In one example the sample is at least one cell biopsied or otherwise removed from an embryo, such as an in vitro fertilization-derived embryo or pre-embryo. In another example, the sample contains fetal cells isolated from maternal blood. In a further example, the sample is a forensic sample.

Samples may also be a biological or non-biological material that contains trace amounts of “contaminating” biological materials. For example, methods described herein are specifically contemplated for use in detecting the presence of bacteria or viruses in a sample such as food, water, drugs, an otherwise inert powder, a package, or other item. Samples include any item that may contain, or be contaminated, with a microbe or infectious agent, particularly a biological agent that could cause disease and/or be used for bioterrorism. Samples also include food or water, or other materials that may contain or be contaminated with a microbe, such as a disease- or illness-causing microbe, and drug preparations, such as those that are prepared using recombinant DNA technology.

Strand displacement activity: The ability of a polymerase to displace a hybridized downstream (non-template) DNA strand encountered during synthesis. Displacement of a DNA strand makes the displaced strand available as template for primer hybridization and DNA replication. Examples of DNA polymerases with strand displacement activity include, but are not limited to, Phi29 DNA polymerase, Bst DNA polymerase, Vent_{R}™ and Deep Vent_{R}™ DNA polymerases, 9° N. DNA polymerase, Klenow fragment of DNA polymerase 1, PhiPRD1 DNA polymerase, phage M2 DNA polymerase, T4 DNA polymerase, and T5 DNA polymerase.

In contrast to polymerases with strand displacement activity, some polymerases (such as Taq DNA polymerase) degrade downstream hybridized DNA encountered during synthesis via a 5′-3′ exonuclease activity.

Target DNA or RNA sequence: Any nucleic acid sequence of interest, for instance sequence(s) intended to be amplified. The target sequence can include multiple nucleic acid molecules (such as a panel of disease-causing genes), multiple sites in a nucleic acid molecule (such as several regions of a particular gene or several genes, or different regions within a genome), or a single region of a nucleic acid molecule. In some applications, such as WGA, the intended target is the entire (that is, whole) genome, but the goal of complete genome amplification is rarely (if ever) realized using conventional technology.

Template nucleic add: A nucleic acid strand that is the substrate for synthesis of a complementary nucleic acid, such as by the annealing of a primer and extension by a DNA polymerase, or by reverse transcribing DNA from an RNA template.

Whole genome amplification (WGA): Methods designed to generate multiple copies of genomic DNA from a sample, with the intent (that is usually not realized) of amplifying all or substantially all of the genome. Particular examples of WGA methods include ligation-mediated PCR, degenerate oligonucleotide primed PCR (DOP-PCR), primer extension preamplification PCR (PEP-PCR), and multiple displacement amplification (MDA). Ligation-mediated PCR WGA is based on digesting the DNA with a restriction enzyme, ligating adaptor sequences to the digested DNA, and then using primers complementary to the adaptor sequences to amplify the DNA (see Saunders et al. Nucl. Acid Res. 17:9027-9037, 1989). DOP-PCR uses a set of primers with a random 3′ end and a fixed 5′ end. The primers anneal throughout the DNA sequence and extended by a DNA polymerase. Primers complementary to the fixed sequence are then used to amplify the sequences (see U.S. Pat. No. 5,731,171). PEP-PCR uses a set of random primers to directly prime DNA amplification by PCR, followed by a second PCR reaction using sequence-specific primers (see U.S. Pat. No. 6,365,375).

In contrast, MDA does not use PCR, or any other temperature cycling-based amplification, in order to generate copies of genomic DNA. MDA uses a set of random primers and a DNA polymerase with strand displacement activity, such as Phi29 DNA polymerase, or other polymerase with strand displacement activity. As the strand displacement polymerase extends the random primers, when it encounters another strand of replicating DNA, or a double-stranded DNA template, it displaces the complementary strand from the template. This makes additional sites for priming and replication available and results in the generation of highly branched replicating structures (see, for example, U.S. Pat. Nos. 6,124,120 and 6,977,148).

### III. Discussion of Certain Related Technologies

A number of techniques have been developed to accomplish whole genome amplification from samples as small as a single cell. These include PCR-based methods, such as ligation-mediated PCR, degenerate oligonucleotide primed PCR (DOP-PCR), and primer extension preamplification PCR (PEP-PCR), and non-PCR-based methods, such as multiple displacement amplification (MDA).

One technique that has been used for WGA is ligation-mediated (or linker-adapter) PCR (see Saunders et al., Nucl. Acid Res., 17:9027-9037, 1989). In this technique, the starting genomic DNA is fragmented, for example by restriction endonuclease digestion or mechanical shearing. An adapter sequence is subsequently ligated to the ends of the DNA fragments. These adapter sequences can be used for amplification of the DNA by standard PCR, using primers complementary to the adapters. While this method can be used to amplify DNA from small amounts of starting material, it has several drawbacks. First, the resulting amplified DNA is of low molecular weight (up to about 2 kb). This prevents analysis of larger regions of DNA, for example, when a gene deletion may be present. This also increases the risk that a sequence of interest will not be present in its entirety in an amplified fragment, making PCR-based analysis impossible. Second, ligation-mediated PCR does not provide an unbiased representation of the genome. For example, in analyzing 23 sequence-tagged sites from DNA amplified by this technique, some samples showed as many as 57% sites that could not be detected (Liu et al. Diagn. Mol. Pathol. 13:105-115, 2004). Thus, ligation-mediated PCR methods of WGA do not provide for reliable amplification of sequences of interest.

Another PCR-based WGA method is degenerate oligonucleotide primed PCR (DOP-PCR) (see Telenius et al. Genomics 13:718-725, 1992; U.S. Pat. No. 5,731,171). DOP-PCR uses a set of primers that have a random 3′ end and a fixed 5′ sequence. The random portion of the primer should anneal evenly throughout the DNA sample and be extended by a polymerase. Following extension, the products are amplified by standard PCR using primers that target the fixed sequence. This technique also has several drawbacks, including small product size and high rates of allelic dropout. The average size of amplification products from DOP-PCR WGA is about 500 base pairs (Cheung and Nelson, Proc. Natl. Acad. Sci., 93:14676-14679, 1996); like DNA produced by ligation-mediated PCR, this may be too small for many desired downstream analysis methods. Similarly, DOP-PCR amplified DNA does not show an unbiased representation of the starting material, resulting in frequent failure to detect sequences of interest following amplification.

Primer extension preamplification PCR (PEP-PCR) is a third PCR-based WGA technique (see Zhang et al., Proc. Natl. Acad. Sci., 89:5847-5851, 1992; U.S. Pat. No. 6,365,375). PEP-PCR uses a set of random primers and a DNA polymerase to amplify the whole genome by multiple rounds of primer extension. A set of sequence-specific primers is then used in a subsequent PCR reaction to amplify a target sequence. Like other PCR-based WGA methods, PEP-PCR has the drawbacks of producing low molecular weight DNA (about 1 kb or less) and incomplete coverage of the genome. For example, it is estimated that PEP-PCR produces less than 30 copies of 22% of the genome (Zhang et al., Proc. Natl. Acad. Sci., 89:5847-5851, 1992), increasing the likelihood that a specific sequence of interest may not be detected following this method of WGA.

A method of WGA that is not based on PCR has been developed (U.S. Pat. Nos. 6,124,120 and 6,977,148; Dean et al., Proc. Natl. Acad. Sci., 99:5261-5266, 2002). This technique, called multiple displacement amplification (MDA) utilizes a DNA polymerase that has high processivity and strand displacement activity, unlike the polymerases used in most PCR-based techniques. MDA employs short random primers to prime DNA replication throughout the genome using a polymerase such as Phi29 DNA polymerase. When this polymerase reaches a double-stranded region of DNA, it displaces the complementary strand, allowing DNA synthesis to continue. In addition to allowing the synthesis of high molecular weight DNA (averaging about 10 kb), this mechanism makes the displaced strand available for further priming events and the replication of additional amounts of DNA. Although MDA using a polymerase such as Phi29 polymerase reduces the problem of low molecular weight DNA products, it still suffers from amplification bias, resulting in incomplete coverage of the genome. This is particularly apparent when starting with very small amounts of DNA. When larger amounts of DNA are used, such as nanogram amounts of DNA, less representation bias occurs (see Dean et al., Proc. Natl. Acad. Sci., 99:5261-5266, 2002). The results are less favorable when the starting material is a single cell (a single diploid mammalian cell contains about 6 pg of DNA). For example, a study that attempted to “optimize” MDA conditions for WGA from single cells observed an average loss of about 26% of the alleles examined (Spits et al., Hum. Mutat. 27:496-503, 2006). Thus, biased representation (allelic dropout) remains a major obstacle to reliable WGA from a single cell.

### IV. Overview of Several Embodiments

Target-oriented whole genome amplification (TOWGA) is a method that insures that specific sequence(s) of interest will be included in the resulting amplification products. A target toward which the TOWGA amplification is directed includes any specific sequence(s) desired to be preferentially included in the amplification product, for example a sequence that is intended to be subject to additional, subsequent analysis. In general, TOWGA includes incubating a mixture containing at least (1) a sample containing nucleic acid template (e.g., DNA or RNA), (2) random primers, (3) a primer designed to be complementary to a target sequence, and (4) a polymerase (particularly one capable of strand displacement replication) under conditions that promote nucleic acid (e.g., DNA or RNA) replication (polymerization). TOWGA also encompasses methods that provide simultaneous preferential targeting and amplification of multiple sequences of interest, by including more than one primer, each designed to be complementary to a target sequence (usually each of which is a different sequence), in the amplification reaction.

Provided herein are methods of target-oriented whole genome amplification (TOWGA), which are methods of amplification targeted to (that is, oriented towards or biased towards) a specific sequence of interest in the context of WGA by multiple strand displacement. TOWGA methods are based on the addition of at least one sequence-specific primer in a reaction that amplifies the whole genome by MDA. TOWGA is particularly useful for the reliable amplification of sequences of interest by WGA from samples containing very small amounts of DNA (e.g., picogram amounts).

Representative TOWGA methods include mixing a sample (containing a template nucleic acid), a set of primers containing random primers and at least one sequence-specific primer, and a polymerase (e.g., DNA or RNA polymerase), and incubating the mixture under conditions that promote nucleic acid replication by strand displacement amplification. In some examples, for example when the target sequence is a viral RNA sequence, RNA can be reverse transcribed into DNA, and the resulting DNA used in the methods provided herein. Examples of the described methods include mixing a sample, a set of primers containing random primers and multiple sequence-specific primers targeting multiple sequences of interest, and a DNA polymerase, and incubating the mixture under conditions that promote DNA replication by strand displacement amplification. Examples of the described amplification methods also include repeating TOWGA amplification on a sample, utilizing DNA replicated in a first TOWGA reaction as the starting material for another round of target-oriented amplification. This repeated method can be used to generate additional quantities of amplified DNA. In the second (or subsequent) round(s) of target-oriented amplification, usually the same sequences are targeted, or a subset thereof. However, this is not essential. Another example of the described amplification methods includes TOWGA utilizing DNA replicated in a preliminary WGA reaction (i.e. a MDA reaction containing only random primers) as the starting material for at least one round of target-oriented amplification. The optional preliminary WGA reaction can be used to generate additional quantities of DNA, for instance, if genomic DNA from a single cell subsequently is to be divided among two or more parallel TOWGA reactions.

In a specific embodiment, the DNA polymerase includes a polymerase with strand displacement activity, such as a Phi29 DNA polymerase.

In another specific embodiment, the conditions that promote replication are substantially isothermal, that is, thermal cycling conditions are not used. Conditions that support replication are largely dependent upon the polymerase (or mixture of polymerases) utilized. It will be recognized in the art that replication conditions can be tailored to support polymerization by the specific DNA polymerase (or combination thereof) used in the amplification reaction.

In another specific embodiment, the random primers and/or the sequence-specific primers contain at least one modified nucleotide. Specifically contemplated herein are modification(s) such that the primers are rendered relatively more resistant to 3′-5′ exonuclease activity than primers containing unmodified nucleotides. In one specific example, at least one of the primers contains at least one thiophosphate-modified nucleotide (such as 1, 2, 3, 5, 10 or 15 thiophosphate-modified nucleotides).

In a further embodiment, the primers may be present at varying concentrations with respect to one another. For example, the random primers may be present in a 50-fold excess compared to a sequence-specific primer. However, when multiple sequence-specific primers are present simultaneously, it is not required that each of the sequence-specific primers be present at the same concentration relative to each other.

In further specific examples, the sample can include a single cell, such as an embryo biopsy, or a sample prepared from a single cell, such as a nucleus. The sample can also include samples with a low number of cells, such as forensic samples, or samples that may contain (or are suspected of containing) an infectious agent or a bioterrorism agent. In another example the sample can include DNA that has been generated by a previous TOWGA reaction.

Details of specific aspects of target-oriented whole genome amplification are provided below. It will be recognized that the discussion below is intended to provide representative examples and is not limiting.

### V. DNA Replication Conditions and Polymerases

Replication of a nucleic acid molecule (e.g., DNA or RNA) by target-oriented amplification methods described herein can include at least one polymerase; the reaction mixture is incubated under conditions that promote nucleic acid replication, and usually such conditions are tailored for the polymerase (or mixture of polymerases) used in the amplification reaction. Replication of DNA by target-oriented amplification methods described herein involve at least one DNA polymerase; the reaction mixture is incubated under conditions that promote DNA replication, and usually such conditions are tailored for the polymerase (or mixture of polymerases) used in the amplification reaction. This section provides representative and non-limiting examples of polymerases and DNA replication (polymerization) conditions. One skilled in the art will appreciate that similar methods with the appropriate modifications can be used for RNA amplification, for example when the target is a viral RNA sequence.

In certain examples, conditions that promote replication are substantially isothermal (that is, the conditions do not include thermal cycling). The appropriate temperature or temperature range for DNA replication is largely influenced by the DNA polymerase (or mixture thereof) chosen. Mesophilic DNA polymerases (including Phi29, T4, T7, and DNA polymerase I) have maximal activity at about 25-40° C. The thermophilic DNA polymerases (such as Taq, Bst, 9° Nₘ, Vent_{R}, and Deep Vent_{R} DNA polymerases) are maximally active at about 75-85° C.

Replication conditions for some amplification reactions optionally include a denaturing step, followed by isothermal incubation during which DNA polymerization occurs. By way of example, a relatively short (e.g., a few minutes, such as 3, 4, or 5 minutes) denaturation at 95° C. is followed by a longer (e.g., several hours, such as about 16 hours) polymerization/amplification phase at a temperature appropriate for the polymerase(s) used (e.g., about 30° C. for Phi29 DNA polymerase). In other examples, no denaturing step is used.

The DNA polymerase used for TOWGA can be a polymerase with strand displacement activity, or a mixture of polymerases at least one of which has strand displacement activity. A DNA polymerase with strand displacement activity is one that has the ability to displace, either alone or in combination with a strand displacement factor, a hybridized strand encountered during replication. DNA replication initiated at a site of primer hybridization will extend to and displace strands being replicated from primers hybridized at an adjacent site. Displacement of an adjacent strand makes it available for hybridization to another primer and subsequent initiation of additional DNA replication. In some examples, the DNA polymerase is highly processive, in order to generate high molecular weight DNA. In some examples, the DNA polymerase lacks 5′-3′ exonuclease activity, which if present, may result in degradation of newly synthesized strands. Examples of DNA polymerases with strand displacement activity include, but are not limited to, Phi29 DNA polymerase, Bst DNA polymerase, Vent_{R}™ and Deep Vent_{R}™ DNA polymerases, 9° N. DNA polymerase, Klenow fragment of DNA polymerase PhiPRD1 DNA polymerase, phage M2 DNA polymerase, T4 DNA polymerase, and T5 DNA polymerase. A particular exemplified strand displacement polymerase is Phi29 DNA polymerase.

### VI. Primers

Methods described herein use a mixture of primers (also referred to as a set of primers) that includes a collection of more than one primer, and usually many different primers. A set of primers for TOWGA includes random primers (usually many random primers of different sequences) and at least one sequence-specific primer that is designed to be complementary to a portion of a target sequence. Random primers will anneal “randomly” throughout the entire genome and prime relatively non-specific replication, allowing for global amplification of DNA present in the sample. The presence of one or more sequence-specific primers permits targeted amplification of sequences of interest. The sequence-specific primer(s) anneal to their respective complementary sequence in the sample and prime replication of the target sequence. This insures that specific sequences of interest will be represented in the amplification product, preventing their potential dropout, as can occur in non-target-oriented WGA methods.

Amplification that maintains a sequence of interest in the resultant amplification product is accomplished by adding a single sequence-specific primer to a multiple displacement amplification reaction, which otherwise would contain only random primers. If only a single specific sequence is being targeted, only a single target primer needs to be included in the TOWGA reaction; there is no need for paired primers for a target sequence. A sequence-specific primer may be complementary to a sequence located either upstream or downstream of the sequence (target) that is of interest (for instance, that will be subsequently analyzed), and it may be complementary to the coding or non-coding strand of the target sequence. If multiple sequences are targeted, multiple sequence-specific primers can be added to the reaction simultaneously. In any instance, however, there need only be one sequence specific primer for any target sequence.

TOWGA can be carried out with a set of primers that includes random primers and at least two different sequence-specific primers (such as at least 3, at least 5, at least 10, at least 25, at least 30, at least 40, or at least 50 different sequence-specific primers, for example 2-35 different sequence-specific primers, 5-25 different sequence-specific primers, 10-15 different sequence-specific primers, and so on). In a particular example, the TOWGA reaction can include primers specific for sequences from 15 different genes. TOWGA can include at least two sequence-specific primers complementary to different genes or other sequences of interest, multiple regions of a single gene or of several genes or sequences, or a single region of a nucleic acid molecule.

### A. Random Primers

A TOWGA reaction includes a set of primers that includes random primers. Random primers will be complementary to sequences distributed throughout the template. The phrase “random primers” as used herein specifically contemplates a collection of individual oligonucleotides of different sequences, for instance which can be indicated by the generic formula 5′-XXXXXX-3′, wherein X represents a nucleotide residue that was added to the oligonucleotide from a mixture of a definable percentage of each dNTP. Sets of primers having random sequences can be synthesized by standard techniques that allow the addition of any nucleotide at each position. The positions of alignment and binding (annealing) of random primers to a template nucleic acid molecule are substantially indeterminate with respect to the template under conditions wherein the primers are used to initiate polymerization of a complementary nucleic acid.

In one example, the random primers used in an amplification reaction are at least six nucleotides in length. In a further example, the random primers are longer, for instance, at least ten nucleotides in length, at least fifteen nucleotides in length, and so forth. In a particular embodiment the random primers are six nucleotides in length. Also contemplated are random primer sets that include primers of different length; ranges in such a set may be, for instance, about 5-20 nucleotides, about 5-15 nucleotides, or about 5-10 nucleotides.

### B. Sequence-Specific Primers

A TOWGA reaction includes a set of primers that contains at least one sequence-specific primer. Sequence-specific primers are designed to have a sequence complementary to a specific sequence of interest and will hybridize to the target sequence and prime DNA replication under appropriate conditions. In some examples, sequence-specific primers are located 5′ to the sequence of interest, for example within at least 10 kb of the target. In another example, sequence-specific primers are within at least 5 kb, at least 1 kb, at least 500 bp, at least 100 bp, or at least 50 by of the sequence of interest.

The sequence-specific primer can be any length that supports specific and stable hybridization between the primer and the target sequence. In one example the primer can be 5 to 60 nucleotides in length. In another example, the primer is 10 to 50 nucleotides in length. In a particular example, the primer is from 15 to 30 nucleotides in length. In a further example, the sequence-specific primer is 20 to 23 nucleotides in length.

### C. Primer Modifications

Primers for use in TOWGA can include one or more modified nucleotides. In various examples, random primers, the sequence-specific primer(s), or both are modified so that the linkage between at least two nucleotides includes a phosphate modification, such as a thiophosphate molecule. Thiophosphate-modified primers are more resistant to degradation by 3′-5′ exonucleases than unmodified primers. Other modifications that can be used for this purpose include phosphorothioate, chiral phosphorothioate, phosphorodithioate, phosphotriester, aminoalkylphosphotriester, methyl and other alkyl phosphonates (including 3′-alkylene phosphonate and chiral phosphonates), phosphinates, phosphoramidates (including 3′-amino phosphoramidate and aminoalkylphosphoramidates, thionophosphoramidates, thionoalkylphosphonates, thionoalkylphosphotriesters, and boranophosphates). These phosphate or modified phosphate linkages can be at or between nucleotides at 2′-5′ or 3′-5′ positions. The linkages can have both polarities such as 2′-5′ and 5′-2′ or 3′-5′ and 5′-3′.

By way of example, the following United States patents describe nucleotides containing modified phosphates: U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Other modifications that enhance oligonucleotide resistance to nucleases may also be used. These include 2′-O-substituted nucleotides, such as 2′-O-methyl alkylated nucleotides (see U.S. Pat. No. 5,914,396 by way of example). The phosphodiester backbone between nucleotides may also be replaced with an amide linkage, such as N-(2-amino-ethyl)-glycine (see U.S. Pat. Nos. 5,539,082 and 5,637,683).

### D. Relative Primer Amounts

As discussed herein, TOWGA reactions contain a set of primers including random primers and at least one sequence-specific primer designed to be complementary to a target DNA sequence. In particular examples, the set of random primers is present in excess compared to the specific primer(s) in the reaction, for instance in about 50:1, 25:1, or 12.5:1 molar excess compared to each (or at least one) sequence-specific primer present in the reaction. In a further example, the set of random primers is present in about equimolar amounts with each (or at least one) sequence-specific primer present in the reaction. It is not necessary that the sequence-specific primers be present at the same concentrations relative to each other, therefore the ratio of random primer to sequence-specific primer may be different for each sequence-specific primer present in the reaction. Nor is it necessary or expected that each random primer within a set will be present in equimolar amounts compared to other random primers.

Although the set of random primers as a whole is usually present in molar excess, or at least equimolar amounts, compared to the sequence-specific primer(s) in the TOWGA reaction, each sequence-specific primer will be present in excess compared to any individual random primer in the reaction.

### VII. Samples

The sample is any sample containing nucleic acids that can be used as starting material (template) for amplification. In essence, any nucleic acid sample would be appropriate, though the provided amplification methods are particularly beneficial for use with highly complex samples (such as genomes).

In general, the sample does not need any unusual preparation prior to its use for amplification using TOWGA. If the sample contains intact cells, lysis of the cells to provide access to the nucleic acids can be beneficial, and may accomplished using art known methods such as by use of a lysis buffer, or by incubation in water (that is by osmotic pressure) for certain cell types. The TOWGA method is particularly suited to samples that contain a small amount of starting material, such as a few cells or a single cell. In one example, the sample is a single cell, or is prepared or extracted from a single cell, such as a nucleus. In a particular example, the sample is a nucleus isolated from a single neuron. In another example, the sample is a single cell from an embryo or pre-embryo, such as an embryo or pre-embryo created by in vitro fertilization. In a further example, the sample can be fetal cell(s) that have been isolated from maternal blood, such as isolation of fetal nucleated red blood cells by cell sorting using a fetus-specific marker, or cells obtained by amniocentesis. The sample can also be a forensic sample, such as blood, saliva, semen, urine or feces, hair, skin, bone, or other body tissues.

In some embodiments, a sample contains between about 1 fg and about 50 ng of nucleic acid (e.g., genomic DNA or viral RNA); for example, between about 5 fg and about 50 ng, between about 10 fg and about 1 ng, between about 10 fg and about 50 pg, between about 0.1 pg and about 50 pg, between about 0.1 pg and about 10 pg, or between about 1 pg and about 10 pg.

Methods are also provided for amplification of a target sequence of interest by one or more successive rounds of TOWGA. In such methods, DNA amplified in a first TOWGA reaction can be used as starting material for a successive TOWGA reaction, where the TOWGA-replicated DNA is mixed with a set of primers containing random primers and a sequence-specific primer and a DNA polymerase and incubated under conditions that promote DNA replication. In a particular example, one successive round of TOWGA is carried out following the initial reaction.

### VIII. Analysis of Replicated DNA

TOWGA generates amplified nucleic acids (e.g., DNA) that includes both sequences targeted by the included sequence-specific primer(s) and other nucleic acid sequences that were replicated from the included random primers. Following one or more rounds of TOWGA, the amplified nucleic acid (e.g., the specific or general amplified DNA or RNA, or both) can be subjected to one or more types of additional analysis. Such analysis may be for myriad different reasons, including for instance detecting the presence or absence of a mutation or polymorphism; detecting gene rearrangements or aneuploidy; gene profiling, such as for the purpose of establishing identity, relatedness, and so forth; determining the presence or absence of an infectious agent or contaminating DNA or RNA, such as from a microbial agent; or for other purposes that will be recognized by one of ordinary skill.

It is particularly contemplated that subsequent analysis will focus on the sequence(s) of interest targeted by the inclusion of sequence-specific primers in the TOWGA reaction. However, it is understood that analysis of any sequence that was present in the starting material may also be carried out, as replication from the random primers produces amplification of the whole genome. To be certain that a target of interest is present in the amplified sample, at least one primer specific for that sequence can be added to the reaction. It is believed that non-targeted sequences will be subject to some level of allelic drop out or other bias that is inherent in multiple displacement amplification WGA, and more generally that non-targeted sequences will be less reliably amplified. Long sequences are amplified much worse compared to shorter sequences when there are no sequence specific primers.

The analysis of nucleic acid (e.g., DNA) includes techniques that are well known in the art. Many, but not all techniques for analysis of DNA include the use of PCR. For example, particular methods of analysis include, but are not limited to, restriction fragment length polymorphism (RFLP), single strand conformational polymorphism (SSCP) mapping, direct nucleic acid sequencing, hybridization, fluorescent in situ hybridization (FISH), comparative genome hybridization (CGH), DNA microarrays, pulsed field gel electrophoresis (PFGE) analysis, RNase protection assay, allele-specific oligonucleotide (ASO), dot blot analysis, allele-specific PCR amplification (ARMS), oligonucleotide ligation assay (OLA) and PCR-SSCP. Methods of performing such methods are routine. See, for example, Chapters 5, 6 and 17 in Human Molecular Genetics 2. Eds. Tom Strachan and Andrew Read. New York: John Wiley & Sons Inc., 1999.

In a particular example, subsequent analysis includes amplification of at least one target sequence by PCR, for instance to detect the presence (or absence) of amplified DNA of the expected length. In another example, the sequence of the replicated target sequence can be determined by direct nucleotide sequencing. In a further example, the presence of a mutation or polymorphism can be detected by ARMS, OLA, or PCR-SSCP analysis. In another example, replicated DNA can be analyzed by Southern blotting or CGH.

### IX. Kits

Kits can be constructed for TOWGA use in various applications, such as pre-implantation genetic diagnosis (PGD), forensic testing, or testing for microbial agents, infectious agents or biological contamination for identification of microbial agents (such as bacteria, viruses, fungi, parasites, and so forth). Such kits can contain basic reagents for use with TOWGA (such as random primers, a strand displacement DNA polymerase, and appropriate buffers) and may optionally include one or more sequence-specific primers. The sequence-specific primers can be tailored for particular applications.

For example, for PGD, kits can contain a panel of sequence-specific primers for use in the TOWGA reaction, which sequence specific primers provide for targeted amplification of recognized congenital disease-causing genes. Such primers can include those that would amplify known disease-causing genes such as cystic fibrosis, Huntington's disease, Tay-Sachs disease, sickle cell anemia, Duchenne muscular dystrophy, β-thalassemia, spinal-muscular atrophy type 1, hemophilia and Fragile X syndrome, though this is not an exhaustive list.

Kits for forensic testing can include sequence-specific primers for use in TOWGA that provide for targeted amplification of genetic markers used for identity testing. The sequence-specific primers can include a panel that would provide amplification of a number of short tandem repeats (STR) or variable number tandem repeats (VNTR) that can be used to determine identity. A panel of sequence-specific primers for identity testing can include primers that would provide amplification of markers known in the art such as D7S820, D13S317, D5S818, FGA, vWA, D3S1358, D18S51, D21S11, D8S1179, TPOX, CSF1PO, THO1, D16S539, and amelogenin.

Kits for testing for biological (e.g., microbial, such as bacterial or viral) agents, infection, or contamination can include sequence-specific primers that amplify DNA sequences specific to known microbes, such as bacteria or viruses. For example, a panel of sequence-specific primers for detection of food- or water-borne microbial contaminants can include primers that would provide amplification of sequences known in the art to be specific to organisms such as E. coli, Campylobacter, Salmonella, hepatitis A, Norwalk-like viruses, or Cryptosporidia. A panel of sequence-specific primers for detection of potential bioterrorism agents can include primers that would provide amplification of sequences known in the art to be specific to agents such as Bacillus anthracis (anthrax), Variola major (smallpox), Salmonella typhi (typhoid fever), Yersinia pestis (plague). Further examples of microbial agents that could be detected with panels of sequence-specific primers can include, but are not limited to, influenza virus, human immunodeficiency virus, West Nile virus, Lyme disease (Borrelia burgdorferi), tuberculosis (Mycobacterium tuberculosis), malaria (Plasmodium), trypanosomes (Leishmania, Trypanosoma brucei), babesiosis (Babesia), and Candida.

### EXAMPLES

The following examples are provided to illustrate certain particular features and/or embodiments. These examples should not be construed to limit the invention to the particular features or embodiments described.

### Example 1

### Target-Oriented Whole Genome Amplification

This example describes a method for amplification of a specific target sequence by multiple displacement amplification. The method is referred to as target-oriented whole genome amplification, or TOWGA. In this method, random and sequence-specific primers are included in a multiple displacement amplification reaction in order to achieve reliable amplification of specific target sequences from a single nucleus. Although particular samples, targets, and reaction conditions are described, one skilled in the art will appreciate that these methods can be appropriately modified for the sample and target nucleic acid of interest.

Nuclei from single neurons in brain slices from mice carrying an L1 element transgene with a green fluorescent protein (GFP) marker were removed with a micropipette and transferred to a 0.2 ml tube containing 5 μl of distilled water. Five (5) μl of a mixture containing 50 mM Tris-HCl, pH 7.5, 100 mM KCl, 20 mM MgCl₂, 200 μM thiophosphate-modified random hexamer primers, and 4 μM, 8 μM, or 16 μM thiophosphate-modified sequence-specific primer was added. The sequence-specific primers were directed to the 5′ and 3′ ends of the L1 element, and had the following sequences, respectively: 5′-AAAGACCCCAACGAGAAGCG-3′ (SEQ ID NO: 1) and 5′-CCTATTGGCGTTACTATGGGAAC-3′ (SEQ ID NO: 2). The mixture was heated at 95° C. for 3 minutes. Then, 10 μl of a mixture containing 49 mM Tris-HCl, pH 7.5, 50 mM KCl, 20 mM MgCl₂, 10 mM (NH₄)₂SO₄, 2 mM dNTPs, 100 μM thiophosphate-modified random hexamer primers, 2 unit/ml yeast pyrophosphatase, and 1600 units/ml Phi29 DNA polymerase was added. The mixture was incubated for 16 to 18 hours at 30° C.

Sequences of interest amplified by TOWGA were further analyzed by standard PCR. Following TOWGA, PCR amplifications were performed in a total volume of 50 μl containing 25 μl of buffer D (Epicentre), FailSafe polymerase (Epicentre), 100-200 ng of each oligonucleotide primer, and 50 ng of DNA obtained in the TOWGA reaction. In order to identify the presence of the L1 transgene, PCR was carried out with GFP968F (5′-GCACCATCTTCTTCAAGGACGAC-3′, SEQ ID NO: 3) and GFP1013R (5′-TCTTTGCTCAGGGCGGACTG-3′, SEQ ID NO: 4) primers, as described in Ostertag et al. (Nucl. Acid Res., 28:1418-1423, 2000). A touch-down PCR protocol was used consisting of: 4 minutes at 94° C.; 20 cycles of 45 seconds at 94° C., 45 seconds at 68° C. decreasing by 0.5° C. per cycle, 1.5 minutes at 68° C.; 35 cycles of 45 seconds at 94° C., 45 seconds at 55° C., 1.5 minutes at 68° C.; 10 minutes at 68° C. PCR products were analyzed by electrophoresis on a 1% agarose gel.

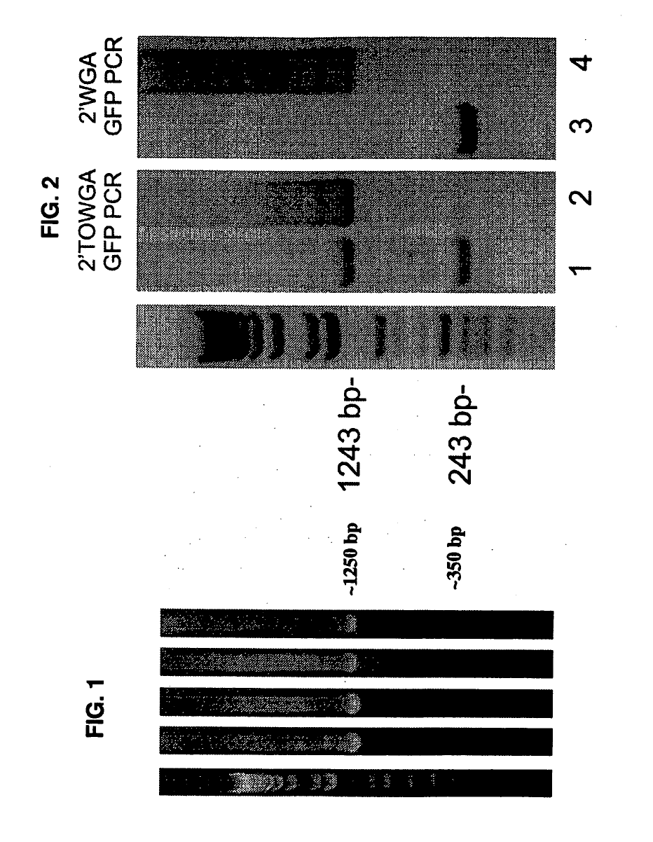

As shown in FIG. 1, the spliced and/or unspliced form of GFP, which is included in the L1 transposon construct, was detected in the TOWGA amplified product. Thus, TOWGA led to the successful detection of the L1 construct in genomic DNA from single mouse neurons.

### Example 2

### Use of Sequence-Specific Primers Alone does not Provide Detectable Amplification

This example illustrates that targeted sequences are not detected using multiple displacement amplification (MDA) carried out using sequence-specific primers alone. This result clearly demonstrates an advantage of combining random and sequence-specific primers in a TOWGA method.

Nuclei from single neurons in brain slices from mice carrying an L1 element transgene with a green fluorescent protein (GFP) marker were removed with a micropipette and transferred to a 0.2 ml tube containing 5 μl of distilled water. Five (5) μl of a mixture containing 50 mM Tris-HCl, pH 7.5, 100 mM KCl, 20 mM MgCl₂, and 4 μM, 8 μM, or 16 μM thiophosphate-modified sequence-specific primer was added. Thiophosphate modified primers directed to the 5′ and 3′ ends of the L1 element as described in Example 1 were used (SEQ ID NOS: 1 and 2). The mixture was heated at 95° C. for 3 minutes. Then, 10 μl of a mixture containing 49 mM Tris-HCl, pH 7.5, 50 mM KCl, 20 mM MgCl₂, 10 mM (NH₄)₂SO₄, 2 mM dNTPs, 2 unit/ml yeast pyrophosphatase, and 1600 units/ml Phi29 DNA polymerase was added. The mixture was incubated for 16 to 18 hours at 30° C. PCR using the GFP-specific primers, as described in Example 1 (SEQ ID NOS: 3 and 4), was used to analyze the TOWGA mixture for the presence (or absence) of the L1 transgene.

When MDA was carried out in the absence of random primers (i.e. using only sequence-specific primers in the reaction), as described in this example, no detectable GFP amplification product was obtained following PCR.

### Example 3

### Comparison of Target-Oriented Multiple Displacement Amplification and Standard Whole Genome Amplification

In this example, TOWGA, which involves the use of random and sequence-specific primers, is compared with WGA, which uses only random primers. This example demonstrates the increased reliability of TOWGA as compared to WGA.

### Materials and Methods

A goal of this example was to amplify by TOWGA and WGA DNA from the same single nucleus. Because a single nucleus includes only a single set of chromosomes, it is not feasible to equally divide such a sample to provide comparable starting material for parallel TOWGA and WGA reactions. Thus, DNA from single nuclei was first amplified using standard WGA amplification (i.e. by multiple displacement amplification using Phi29 DNA polymerase). Briefly, nuclei from single neurons in brain slices from mice carrying an L1 element transgene with a green fluorescent protein (GFP) marker were removed with a micropipette and transferred to a 0.2 ml tube containing 5 μl of distilled water. Five (5) μl of a mixture containing 50 mM Tris-HCl, pH 7.5, 100 mM KCl, 20 mM MgCl₂, and 200 μM thiophosphate-modified random hexamer primers was added. The mixture was heated at 95° C. for 3 minutes. Then, 10 μl of a mixture containing 49 mM Tris-HCl, pH 7.5, 50 mM KCl, 20 mM MgCl₂, 10 mM (NH₄)₂SO₄, 2 mM dNTPs, 100 thiophosphate-modified random hexamer primers, 2 unit/ml yeast pyrophosphatase, and 1600 units/ml Phi29 DNA polymerase was added. The mixture was incubated for 16 to 18 hours at 30° C. This reaction is designated the primary WGA (1° WGA) reaction.

One tenth of the primary WGA reaction was used for a second round of amplification, either including sequence-specific primers plus random primers (2° TOWGA) or including random primers only (2° WGA). Thiophosphate-modified primers directed to the 5′ and 3′ ends of the L1 element (as described in Example 1; SEQ ID NOS: 1 and 2)) were used for 2° TOWGA. Except for the addition of sequence-specific primers in the 2° TOWGA, the conditions of the second round of amplification were the same as for the primary WGA.

In order to identify the presence of the L1 transgene, following the 2° TOWGA and 2° WGA reactions, GFP was amplified by standard PCR using the paired GFP-specific primers described in Example 1 (SEQ ID NOS: 3 and 4). PCR amplification conditions were as described in Example 1. The resulting PCR product was analyzed by electrophoresis on a 1% agarose gel.

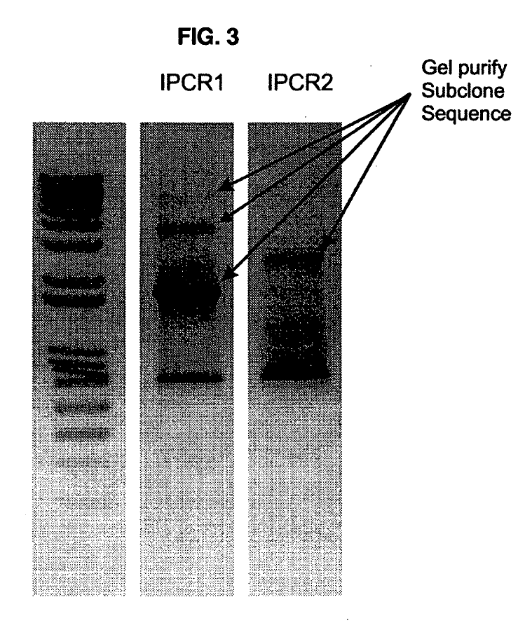

Inverse PCR (IPCR) was used to identify the location of integration of a retrotransposed L1 element on the level of a single cell. The product of the 2° TOWGA (10 μg) was digested with XbaI and ligated overnight at 14° C. under conditions promoting self-circularization. After ligation, DNA was ethanol precipitated and used as a substrate for IPCR. PCR primers to detect the L1 element were the same as the L1 primers described above (SEQ ID NOs: 1 and 2). A touch-down PCR protocol was used and consisted of: 4 minutes at 94° C.; 20 cycles of 45 seconds at 94° C., 45 seconds at 68° C. decreasing by 0.5° C. per cycle, 5 minutes at 68° C.; 35 cycles of 45 seconds at 94° C., 45 seconds at 55° C., 5 minutes at 68° C.; and 10 minutes at 68° C. The resulting PCR product was analyzed by electrophoresis on a 1% agarose gel. IPCR resulted in a number of products shown in FIG. 3. These products were gel purified and sequenced.

### Results

Using comparable starting material and identical detection conditions, FIG. 2 shows that the two expected PCR products, a 1243 by product representing the presence of the L1 element transgene, and a 243 by product representing expressed GFP resulting from integration of the transgene, were detected by 2° TOWGA in genomic DNA from two separate mouse neuron nuclei. However, 2° WGA using genomic DNA from the same two nuclei only amplified the smaller product in one instance (lane 3) and only amplified the larger product in the second independent instance (lane 4).

This example demonstrates that TOWGA, which includes sequence-specific primers in addition to random primers in a multiple strand displacement amplification reaction, more reliably amplifies products of interest from a single neuron than WGA alone.

This example further demonstrates that TOWGA in combination with IPCR successfully identified the position of a retrotransposed L1 element in the Mus musculus NOD-derived CD11c+ve dendritic cells cDNA.

### Example 4

Amplification of Multiple Disease-Related Genes by Multi-Step WGA/TOWGA

This example describes the simultaneous detection of multiple disease-related genes in a sample obtained by the method including a WGA step followed by a TOWGA step. Although particular disease-related targets and reaction conditions are described, one skilled in the art will appreciate that these methods can be appropriately modified for the target nucleic acid molecules of interest.

### Materials and Methods

Nuclei from single neurons in brain slices from mice were removed with a micropipette and transferred to a 0.2 ml tube containing 5 μl of distilled water. Five (5) μl of a mixture containing 50 mM Tris-HCl, pH 7.5, 100 mM KCl, 20 mM MgCl₂, and 200 μM thiophosphate-modified random hexamer primers was added. The mixture was heated at 95° C. for 3 minutes. Then, 10 μl of a mixture containing 49 mM Tris-HCl, pH 7.5, 50 mM KCl, 20 mM MgCl₂, 10 mM (NH₄)₂SO₄, 2 mM dNTPs, 100 μM thiophosphate-modified random hexamer primers, 2 unit/ml yeast pyrophosphatase, and 1600 units/ml Phi29 DNA polymerase was added. The mixture was incubated for 16 to 18 hours at 30° C. One tenth of the product of this 1° WGA was used for 2° TOWGA, as described in Example 3. Six thiophosphate-modified sequence-specific primers complementary to DNA sequences of three different genes of interest were simultaneously included in the 2°TOWGA reaction. The primers were:

| | |
|---|---|
| BRCA 1: | |
| 5′-AAGCCAAAACACATAGACCC-3′ | (SEQ ID NO: 5) |
| and | |
| 5′-CTGCCTGTCGTTACATGTTC-3′; | (SEQ ID NO: 6) |
| MSH-2: | |
| 5′-TGAGCCAGGAAGTGTGTGTG-3′ | (SEQ ID NO: 7) |
| and | |
| 5′-AGGATGGAAGCAGTCTCCAG-3′; | (SEQ ID NO: 8) |
| and | |
| HBA-1: | |
| 5′-TCCCTCACTTTGATGTAAGCC-3′ | (SEQ ID NO: 9) |
| and | |
| 5′-CCAAGAGGTACAGGTGCAAG-3′. | (SEQ ID NO: 10) |

Following TOWGA, PCR amplifications were performed in a total volume of 50 μl containing 25 μl of buffer D (Epicentre), FailSafe polymerase (Epicentre), 100-200 ng of each oligonucleotide primer, and 50 ng of DNA obtained in TOWGA reaction. Primers used in the 2° TOWGA were also used for PCR, however the PCR primers did not include thiophosphate modification. Individual sets of primers directed to a single gene were included in each reaction, i.e. one reaction amplified a portion of BRCA1 using the BRCA1-specific primers (SEQ ID NOs: 5 and 6); one reaction amplified a portion of MSH-2 using the MSH-2-specific primers (SEQ ID NOs: 7 and 8); one reaction amplified a portion of HBA-1 using the HBA-1-specific primers (SEQ ID NOs: 9 and 10). A touch-down PCR protocol was used consisting of: 4 minutes at 94° C.; 20 cycles of 45 seconds at 94° C., 45 seconds at 68° C. decreasing by 0.5° C. per cycle, 1.5 minutes at 68° C.; 35 cycles of 45 seconds at 94° C., 45 seconds at 55° C., 1.5 minutes at 68° C.; 10 minutes at 68° C. PCR products were analyzed by electrophoresis on a 1% agarose gel, purified and sequenced.

### Results

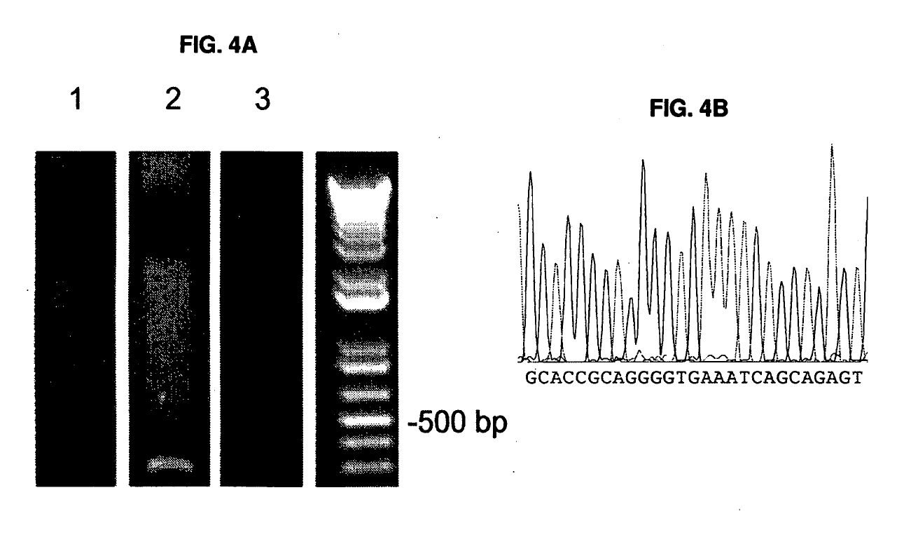

As shown in FIG. 4A, the three genes of interest (mouse BRCA1, HBA1, and MSH2 genes) were successfully detected using genomic DNA from a single mouse neuron nucleus. Sequencing confirmed the uniqueness and specificity of each DNA fragment detected in FIG. 4A. FIG. 4B shows representative sequence obtained from the HBA1 PCR product.

This example demonstrates that sequential WGA and TOWGA reactions provide sufficient amplification of genomic DNA from a single nucleus to simultaneously detect by standard methods, i.e., PCR, multiple target sequences of interest.

### Example 5

### Simultaneous Target Oriented Whole Genome Amplification of Multiple Targets

This example describes the use of TOWGA in a single reaction to simultaneously amplify multiple sequence-specific targets (e.g., genes that may include genetic defects) from one or more template nucleic acid molecules (e.g., genomic DNA or viral RNA) in sufficient amounts to permit detection of such targets by standard methods (e.g., PCR). TOWGA permits, in this example and others described herein, the use of a sample containing very little of the template molecule (such as genomic DNA from a single nucleus or a few nuclei).

A sample containing template nucleic acid molecules is obtained in any known manner consistent with the nature of the sample; for example, nuclei from a single cell or one or more cells of an embryo may be obtained using a micropipette, or a forensic sample may be lifted by swab from a physical object or living subject. Exemplary samples are described throughout this disclosure.

Where template nucleic acid molecules (e.g., DNA or RNA) are not sufficiently accessible to the reagents used in this method of this example to obtain the desired signal, such template molecules can be isolated or partially purified from the sample using methods known to those of ordinary skill in the art. For example, DNA from a forensic sample may be extracted into a small volume of a saline solution (e.g., phosphate-buffered saline), or intact cells may be lysed, for instance, using physical or detergent disruption of the cell membranes Similar methods of obtaining RNA from a sample can be performed using methods known in the art (for example RNA can be isolated from a sample). In some examples, RNA is converted into DNA (e.g. using reverse transcriptase), and the resulting DNA used. The amount of template nucleic acid (e.g., DNA or RNA) in a TOWGA reaction can be considerable; however, TOWGA can amplify sequence-specific targets from very small amounts of template material. Accordingly, although not required, a TOWGA reaction typically will contain less than 100 ng of template nucleic acid molecules. In particular TOWGA reactions, as little as 5-10 pg or even as little as 5-10 fg template nucleic acid molecules can be used. Typically, the template nucleic acid molecules are contained in a relatively small volume of solution amenable to completion of a TOWGA reaction (e.g., the smallest practical volume; for related discussion, see Hutchison et al., Proc. Natl. Acad. Sci., 102(48):17332-17336, 2005). A useful volume of solution in which to conduct a TOWGA reaction may be from about 10 μl to about 50 for example from about 10 μl to about 20 μl; however, other reaction volumes may be suitable. A TOWGA reaction can be carried out in any solution that does not adversely affect the interaction between and/or function of components of the reaction. Most commonly used saline solutions are appropriate, including Tris-buffered salines, phosphate-buffered salines, and HEPES-buffered salines. Exemplary reaction buffers are (i) 25 mM Tris-HCl, pH 7.5, 50 mM KCl, 10 mM MgCl₂, or (ii) 25 mM Tris-HCl, pH 7.5, 25 mM KCl, 10 mM MgCl₂, 5 mM (NH₄)₂SO₄.

The template nucleic acid molecules are mixed in solution with a combination of random and sequence-specific primers. The general natures of such primers are discussed elsewhere in this disclosure. Both random and sequence-specific primers can contain modified nucleotides (such as, thiophosphate-modified nucleotides), for example to substantially reduce primer degradation by 3′-5′ exonucleases.

For the purpose of this example, BRCA1-, MSH-2-, and HBA-1-specific primers, as described in Example 4, are used; however, other sequence-specific primers useful for particular applications, such as pre-implantation genetic diagnosis or forensic analysis are known or may be readily designed. Random primers are as described in any of Examples 1-4, or elsewhere in this disclosure.

Any concentrations of primers and ratio of random:specific primers that results in the desired outcome of simultaneously detecting multiple target sequences of interest may be used. Typically, but not necessarily, the amount (by concentration or by weight) of random primers is greater than the corresponding amount of sequence-specific primers. Exemplary ratios of random primers to each sequence-specific primer are 100:1, 50:1, or 25:1, or as described elsewhere in this disclosure. For this example, the final concentration of random primers in the reaction mixture is about 100 μM, and the final concentration of each sequence-specific primer is between about 1 μM and about 4 μM.

Optionally, the template molecules and all or part (e.g., 50%, 70%, or 80%) of the primer mixture are briefly heated to permit denaturation of the template. A denaturation step is usual in non-isothermal amplification methods, such as PCR, but is not necessary for isothermal, multi-strand displacement amplification (see, e.g., U.S. Pat. No. 6,977,148). The accepted theory is that denaturation increases initial access of the primers to the template. Particular temperatures for and times need to denature template nucleic acid molecules are known in the art. One useful, non-limiting set of conditions for purposes of this example is 95° C. for 3 minutes.

Following (e.g., if using an optional denaturation step) or concurrent with the mixing of the template with random and specific primer, other components of the amplification reaction are added, including dNTPs (to a final concentration (each or collectively) of about 0.1 mM to about 2 mM; e.g., 2 mM), yeast pyrophosphatase (to a final concentration of about 0.1 unit/ml to about 5 units/ml; e.g., 2 units/ml), and Phi29 DNA polymerase (to a final concentration of about 10 units/ml to about 2000 units/ml; e.g., 1600 units/ml). The mixture is incubated for a sufficient time to permit amplification of the template nucleic acid molecule; several hours generally is sufficient (e.g., from about 12 to about 48 hours; such as 16 to 18 hours). The reaction is incubated at a temperature that does not cause significant degradation of the reaction components or unduly slow the reaction (e.g., room temperature, or about 30° C.). Optionally, all or part (e.g., 10%, 25%, or 50%) of the initial (or any subsequent) TOWGA reaction mixture can be further amplified by repeating (once or more) the TOWGA reaction described above.

Following TOWGA, sequences of interest may be further analyzed using conventional techniques, such as PCR amplifications using paired primers specific for each sequence of interest (see, for example, PCR primers described in Example 4). In methods using PCR-based analysis of TOWGA reaction mixtures, PCR products can be analyzed by electrophoresis on a 1% agarose gel, and, optionally, can be purified and sequenced.

This example illustrates a method utilizing one (or more) round(s) of TOWGA to simultaneously amplify multiple target sequences of interest from a minute amount of genomic DNA to a level detectable by standard detection methods.

### Example 6

### Pre-Implantation Genetic Diagnosis

This example describes representative application of TOWGA in pre-implantation genetic diagnosis (PGD). POD is used to screen embryos created by in vitro fertilization for genetic defects prior to embryo transfer to a host. Generally, the parents are known carriers of a particular genetic defect that can be passed to their offspring.

Embryos are generated by standard IVF methods. About three days after IVF, one or two cells are removed from the embryo, for instance by micromanipulation. The cells are lysed and TOWGA is carried out essentially as is described in Example 1, 4 or 5. The reaction contains sequence-specific primers that target region(s) of the genome that may contain a genetic defect inheritable from one of the parents. One or more sequence-specific primers can be included in the TOWGA, for example, to analyze a panel of possible mutations or polymorphisms.

Subsequent to one or more rounds of TOWGA, the amplified DNA is subjected to further analysis, such as sequencing, hybridization, or other techniques, to provide genotyping information. This genotyping allows selection of embryos for implantation.

### Example 7

### Forensic Testing

This example describes representative use of TOWGA to obtain evidence from forensic samples.

A forensic sample is obtained, such as a biological sample or a sample lifted from a non-biological source, which is used then to extract biological material that may be used for the isolation and analysis of DNA or RNA. The biological material, such as tissue, blood, or other body fluids, is collected or collected from a non-biological source using a swab, for instance. The swab is swirled in water or a buffer solution to extract cellular material and optionally the cells are lysed.

TOWGA is carried out as described in Example 1, 4 or 5, except that the selected sequence-specific primers are designed to target regions of DNA that allow identity determination, such as a panel of short tandem repeat regions. Identity of the biological sample is determined by techniques known in the art, such as DNA sizing following PCR with fluorescently labeled primers (such as Applied Biosystems AMPF/STR™ kit).

### Example 8

### Detection of Microbial Nucleic Acid(s)

This example describes representative use of TOWGA to detect microbial nucleic acid by TOWGA, for instance trace amounts that are the result of contamination or infection. This includes detection of food- or water-borne contamination, such as E. coli, Campylobacter, Salmonella, hepatitis A, Norwalk-like viruses, or Cryptosporidia. This also includes the detection of potential bioterrorism agents, such as Bacillus anthracis (anthrax), Variola major (smallpox), Salmonella typhi (typhoid fever), Yersinia pestis (plague), etc. TOWGA can also be used for the detection of other microbial agents, such as infectious agents, including, but not limited to, influenza virus, human immunodeficiency virus, West Nile virus, Lyme disease (Borrelia burgdorferi), tuberculosis (Mycobacterium tuberculosis), malaria (Plasmodium), trypanosomes (Leishmania, Trypanosoma brucei), babesiosis (Babesia), and Candida.

A sample that is suspected to contain a microbial agent is collected. If the sample is food or water, a small amount of the sample is treated so any cells are lysed. If the sample is on a solid substrate (such as an envelope, vial, doorknob, floor, etc.), the sample is collected with a swab which is swirled in water or other liquid to obtain a sample of suspended cells, which are then lysed.

TOWGA is carried out essentially as described in Example 1, 4, or 5, except that primers specific to one or more microbial agents are included in the reaction. Panels of sequence-specific primers can be constructed which include a set of primers for common food- or water-borne contaminants, or a set of primers for potential bioterrorism agents, for instance. Following TOWGA, a second panel of pairs of sequence-specific primers can be used to amplify specific regions by PCR to determine the presence or absence of a particular bacterial or viral agent in the sample.

In view of the many possible embodiments to which the principles of the disclosure and examples may be applied, it will be recognized that the illustrated embodiments are only examples of the invention and are not to be taken as limiting its scope.

We claim:

1. A method of target-oriented whole genome amplification, wherein at least one specific target nucleic acid sequence of a genome is replicated, comprising:
mixing a sample comprising genomic nucleic acid, which comprises at least one specific target nucleic acid sequence, with
a set of primers comprising random primers and at least one sequence-specific primer designed to be complementary to a portion of the target nucleic acid sequence or to a portion of the genomic nucleic acid flanking the target nucleic acid sequence, and
at least one polymerase with strand displacement activity; and
incubating the resultant mixture under conditions that promote replication of nucleic acids to produce an incubated mixture, wherein the at least one specific target nucleic acid sequence is replicated.

2. The method of claim 1, wherein the target nucleic acid sequence comprises a DNA target nucleic acid sequence.

3. The method of claim 1, wherein the at least one polymerase includes Phi29 DNA polymerase.

4. The method of claim 1, wherein the conditions that promote replication are substantially isothermal.

5. The method of claim 1, wherein the at least one sequence-specific primer, the random primers, or both, contain at least one modified nucleotide.

6. The method of claim 1, wherein the set of primers comprises two or more different sequence-specific primers.

7. The method of claim 6, wherein each different sequence-specific primer is designed to be complementary to a portion of a different specific target DNA sequence.

8. The method of claim 1, wherein the random primers are present in about 50:1 molar excess compared to each sequence-specific primer.

9. The method of claim 1, wherein the random primers are present in about 25:1 molar excess compared to each sequence-specific primer.

10. The method of claim 1, wherein the random primers are present in about 12.5:1 molar excess compared to each sequence-specific primer.

11. The method of claim 1, wherein the random primers and each sequence-specific primer are present in about equimolar amounts.

12. The method of claim 1, wherein the random primers are at least six nucleotides in length.

13. The method of claim 1, wherein the sample comprises a single cell or is prepared from a single cell.

14. The method of claim 13, wherein the single cell is from an embryo or a pre-embryo.

15. The method of claim 14, wherein the embryo or pre-embryo was created by in vitro fertilization.

16. The method of claim 1, wherein the sample comprises fetal cells from maternal blood.

17. The method of claim 1, wherein the sample is from a forensic sample.

18. The method of claim 1, further comprising repeating at least once the mixing and incubation steps on all or part of the incubated mixture.

19. The method of claim 1, further comprising subjecting the incubated mixture to further analysis.

20. The method of claim 19, wherein analysis comprises amplification of at least one target DNA sequence by polymerase chain reaction.

21. The method of claim 20, wherein analysis further comprises sequencing at least one target DNA sequence.

22. An improved method for multiple displacement amplification, the improvement comprising adding at least one primer complementary to a specific target DNA sequence or to DNA flanking the specific target DNA sequence to the multiple displacement amplification reaction.

23. An improved method for multiple displacement amplification, comprising
mixing a set of primers with a sample, comprising a template DNA sequence, to produce a primer-sample mixture; wherein the sample comprises a template DNA sequence, which comprises a target DNA sequence,
incubating the primer-sample mixture under conditions that promote hybridization between the primers and the template DNA sequence in the printer-sample mixture,
mixing at least one DNA polymerase with strand displacement activity with the primer-sample mixture, to produce a polymerase-sample mixture, and
incubating the polymerase-sample mixture under conditions that promote replication of the target DNA sequence, wherein replication of the target sequence results in replicated strands, and wherein the set of primers comprises primers having random nucleotide sequences,
the improvement comprising including in the set of primers at least one primer designed to be complementary to the target DNA sequence or to a portion of the template DNA sequence flanking the target DNA sequence.

24. A kit, comprising:
a strand displacement DNA polymerase;
at least two random primers; and
optionally at least one sequence-specific primer.

25. The kit of claim 25, wherein the at least one sequence-specific primer is specific for:
one or more congenital disease-causing genes;
one or more identity markers;
one or more microbial agents; or
combinations thereof.
